(19) United States
(12) Patent Application Publication
(10) Pub. No.: US 2016/0029922 A1
(43) Pub. Date: Feb. 4, 2016

(54) SAMPLING INTERFACES

(71) Applicant: ORIDION MEDICAL 1987 LTD., Jerusalem (IL)

(72) Inventors: Avner Bar-Lev, Givatayim (IL); Alon Sasson, Rehovot (IL); Paul Stanley Addison, Edinburgh (GB); Moshe Mandelbaum, Psagot (IL); Joshua Lewis Colman, Jerusalem (IL)

(21) Appl. No.: 14/447,659

(22) Filed: Jul. 31, 2014

(51) Int. Cl. A61B 5/097; A61B 5/00; A61B 5/1455; A61B 5/083

(52) U.S. Cl. CPC A61B 5/097; A61B 5/0836; A61B 5/6819; A61B 5/682; A61B 5/7264; A61B 5/6843; A61B 5/14551; A61B 5/6838; A61B 5/6833; A61B 5/4839; A61B 5/6831

(57) ABSTRACT

A tubeless patient interface including a grasping member configured to grasp a patient's nose or tooth; and a miniature CO₂ sensor attached to the grasping member and configured to measure the concentration of CO₂ from a patient's breath flow.

### TECHNICAL FIELD

The present disclosure relates generally to the field breath sampling patient interfaces.

### BACKGROUND

Accurate monitoring concentrations of gases, such as for example carbon dioxide (CO₂) in exhaled breath, is vital in assessing the physiologic status of a patient. Breath sampling is generally performed through breath sampling tubes and body interface accessories. The tubing and accessories often cause discomfort for the patient and may in even be an obstacle for the treatment of the patient in certain situations.

Breath sampling may either be diverting (i.e. sidestream) or non-diverting (i.e. mainstream). In diverting breath sampling, the breath sample is transported from the sampling site, through a sampling tube, to the sensor, whereas in non-diverting breath sampling, the breath sample is measured at the sample site.

### SUMMARY

Aspects of the disclosure, in some embodiments thereof, relate to breath sampling interfaces utilizing miniature CO₂ measuring technologies.

Patient breath monitoring in general and CO₂ monitoring in specific, requires sampling patients' breath using tubing and body interface accessories. The tubings and accessories are often causing discomfort to the patient; the degree of discomfort proportional to the size and shape of the interface and to the duration of the monitoring. In certain situations, the tubings and interface accessories are even an obstacle to treatment i.e. when the patient lays on his stomach, is rolled over etc.

The patient interfaces disclosed herein, are advantageously designed to minimize patient discomfort without compromising sampling efficiency and accuracy. For example, by utilizing a miniature CO₂ measuring technology, the patient interface disclosed herein may be a tube-less patient interface, thereby providing a much less cumbersome interface.

The patient interfaces may be configured to interface with the patient at different location on the patient body in so doing enable patients to choose interfaces according to personal preferences.

According to some embodiments, there is provided a tubeless patient interface comprising a grasping member configured to grasp a patients nose or tooth and a miniature CO₂ sensor attached to the grasping member and configured to measure the concentration of CO₂ from a patient's breath flow. According to some embodiments, the interface may be configured to facilitate non-diverted breath sampling. According to some embodiments, the interface may be configured to wirelessly provide the measured CO₂ concentration to a remote control logic.

According to some embodiments, the miniature CO₂ sensor may be a nano-sized optical sensor. According to some embodiments, the miniature CO₂ sensor may be a chemical sensor.

According to some embodiments, the grasping member includes a first and a second arm, and an elastic member configured to connect the first and second arms.

According to some embodiments, the interface is configured to be worn on the patient's nose.

According to some embodiments, the patient interface may include at least two miniature CO₂ sensors. According to some embodiments, the first and second arms may include at least one miniature CO₂ sensor on a proximal end thereof.

According to some embodiments, the proximal ends of the first and second arms are configured to reach underneath or within a patient's nostrils.

According to some embodiments, the stretching of the elastic member may be indicative of the interface being worn by said patient. According to some embodiments, the interface may be configured to on/off patient detection based on the stretching of the elastic member.

According to some embodiments, the elastic member may include an SpO₂ probe.

According to some embodiments, the grasping member may include a circlip configured to grasp a columella of the nose on each side thereof. According to some embodiments, the patient interface may include at least two miniature CO₂ sensors positioned on the ends of the circlip.

According to some embodiments, the grasping member may be a tooth grasping member. According to some embodiments, the tooth grasping member may include a ring structure configured to circumferentially engage a tooth. According to some embodiments, the tooth grasping member may include an adhesive cover configured to be attached to a tooth.

According to some embodiments, the tooth grasping member may include a compartment configured to store therein medicaments. According to some embodiments, the compartment is configured to release said medicaments based on the measured concentration of CO₂.

According to some embodiments, there is provided a patient interface including a grasping member configured to grasp a patient's nose and an elastic member connecting said first and second arms. According to some embodiments, the grasping member may include a first and a second arm. According to some embodiments, the elastic member may be configured to surroundingly engage a nasal ridge of the patient.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 schematically illustrates a tubeless patient interface, according to some embodiments;

FIG. 2 schematically illustrates a tubeless patient interface, according to some embodiments;

FIG. 3 schematically illustrates a tubeless patient interface, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to tubeless patient interfaces of significantly reduced size. The tubeless interfaces disclosed herein are designed to improve patient comfort yet without impairing measurement quality.

According to some embodiments, there is provided a tubeless patient interface including a grasping member a miniature CO₂ sensor.

As referred to herein, the terms “patient” and “subject” may interchangeably be used and may relate to a subject undergoing breath monitoring.

As used herein, the term “patient interface” may refer to a constituent configured to engage a patient (e.g. the patient's nose or mouth), thereby enabling monitoring the concentration of CO₂ in the patient's breath.

As used herein, the term “tubeless” may refer to a patient interface to which no tubes need to be connected in order to facilitate monitoring the concentration of CO₂ in the patient's breath.

As used herein, the term “grasping member” may refer to any component configured to attached, hold, cover or otherwise connect to or be worn by a patient.

According to some embodiments, the interface is configured to grasp a patient's nose or teeth. According to some embodiments, the interface is a nasal interphase. According to some embodiments, a “nasal patient interface” may refer to an interface configured to be worn on a patient's nose. According to some embodiments, a “nasal patient interface” may refer to an interface configured to sample breath from the patient's nose. According to some embodiments, the interface is an oral interphase. According to some embodiments, an “oral patient interface” may refer to an interface configured to be positioned within a patient's mouth. According to some embodiments, an “oral patient interface” may refer to an interface configured to sample breath from the patient's mouth.

According to some embodiments, the miniature CO₂ sensor may be attached to, molded on, glued to, embedded in or otherwise positioned on the grasping member. According to some embodiments, the miniature CO₂ sensor may be attached to the grasping member in such way that it is placed directly in the breath flow of the patient. According to some embodiments, the miniature CO₂ sensor may be configured to measure the concentration of CO₂ directly from the patient's breath. According to some embodiments, the interface may be configured to facilitate non-diverted breath sampling. It is thus understood that no tubes need to be connected to the patient interface, as no breath samples are transferred for analysis, thereby significantly improving patient comfort and mobility.

As used herein the term “non-diverted breath sampling” may refer to breath sampling performed at the sample site e.g. directly from the patients breath flow.

As used herein the term “miniature” when referring to a CO₂ sensor, may refer to a CO₂ sensor having a size of less than 1 cm³, less than 0.5 cm³, less than 100 mm³, less than 10 mm³ less than 1 mm³, less than 0.5 mm³ or less than 100 nm³.

According to some embodiments, the interface may include more than one miniature CO₂ sensor. For example the interface may include 2, 3, 4, 5 or more miniature CO₂ sensor.

According to some embodiments, the miniature CO₂ sensor is a nano-sized optical sensor. According to some embodiments, the nano-sized optical sensor may be a Nano-Opto-Mechanical (NOM) sensor configured to determine the concentration of CO₂ based on a relative position of a nanoparticle within a nano-scale void of the NOM sensor. According to some embodiments, the NOM sensor may include two optical elements configured to guide light beams through the void from opposing sides thereof. According to some embodiments, one of the light beams may pass through a breath sample prior to passing through the void. The light beam passing through the breath sample may be partially absorbed by the CO₂ of the breath sample and as a result thereof the intensity of the light beam, subsequently passing through the void, is reduced. In effect, the location of the nano-particle within the nano-scale void may be altered.

According to some embodiments, the miniature CO₂ sensor is a chemical sensor. According to some embodiments, the chemical sensor is polymer- or heteropolysiloxane based. Advantageously, the chemical CO₂ sensor may have very low energy consumption and can be reduced in size to fit into microelectronic-based systems. According to some embodiments, the chemical CO₂ sensor includes one or more sensor spots (SP). According to some embodiments, the sensor spot may be configured to detect a CO₂ mediated change in pH of a buffer. According to some embodiments, the sensor spot may include a chemical optical sensor utilizing the acidic nature of CO₂ for detection. According to some embodiments, the chemical optical sensor may include a gas-permeable membrane. According to some embodiments, the gas-permeable membrane may include a pH-sensitive luminescence dye immobilized together with a buffer and an inert reference luminescent dye. According to some embodiments, CO₂ permeating into the membrane changes the internal pH of the buffer and thereby the luminescence of the pH-sensitive dye. According to some embodiments, the sensor spot may be attached to an inner surface of any transparent vessel, such as, but not limited to, a sampling tube.

As used herein, the terms “breath sampling tube”, “tube” and “breath sampling line” may refer to any type of tubing(s) or any part of tubing system adapted to allow the flow of sampled breath, for example, to an analyzer, such as a capnograph. The sampling line may include tubes of various diameters, adaptors, connectors, valves, drying elements (such as filters, traps, trying tubes, such as Nafion® and the like).

According to some embodiments, the patient interface may be configured to wirelessly provide the measured CO₂ concentration(s) to a remote control logic. As used herein the term “control logic” may refer to a program a processer or other suitable computing tool configured to receive and analyse the obtained measurements.

According to some embodiments, the grasping member may include a first and a second arm, and an elastic member configured to connect the first and second arms. According to some embodiments, the interface is configured to be worn on said patient's nose. For example, the interface may be configured to be worn by the patient in a manner similar to glasses, such that the elastic member surroundingly engages the nasal ridge at a length thereof. As used herein the term “nasal ridge” may refer to the midline prominence of the nose, extending from the nasal root to the tip (also called the dorsum of the nose).

According to some embodiments, each of the grasping arms may include a miniature CO₂ sensor (such that the interface includes two interfaces). According to some embodiments; the miniature CO₂ sensors may be positioned on a proximal end of each of the grasping arms. According to some embodiments, the proximal ends of the first and second arms are configured to reach underneath or within a patient's nostrils.

As used herein, the terms “proximal” and “proximal end” may refer to the part of the interface closest to the to the patient's breath flow.

According to some embodiments, the miniature CO₂ sensors may be positioned at a certain distance from the proximal end of the arms. As used herein the term “certain distance” may refer to 0.1 cm, 0.25 cm, 0.5, cm, 075 cm or 1 cm from the proximal end of the arm. Each possibility is a separate embodiment.

According to some embodiments, the elastic element may be configured to be stretched when worn by the patient. Accordingly, the stretching of the elastic member may be indicative of the interface being worn by the patient. Similarly, when the elastic member is in its relaxed form it may be indicative of the patient interface not being worn by the patient. According to some embodiments, a change in the stretching of the elastic member may be indicative of the interface being put on (increased stretching) or taken off (reduced stretching). Each possibility is a separate embodiment. Hence, the interface may be configured to provide on/off detection based on the stretching of the elastic member

According to some embodiments, the elastic member may include an SpO₂ probe configured to monitor a patient's 0₂ saturation.

According to some embodiments, the grasping member may include a circlip configured to grasp a columella of the nose on each side thereof. As used herein the term “columella” may refer to the fleshy external end of the nasal septum. According to some embodiments the circlip may be configured to grasp the columella from each side thereof (within each nostril).

According to some embodiments, each proximal end of the circlip may include a miniature CO₂ sensor. According to some embodiments, the proximal end of the circlip may refer to part of the circlip closest to the nostrils of the patient.

According to some embodiments, the miniature CO₂ sensors may be positioned at a certain distance from the proximal end of the circlip. As used herein the term “certain distance” may refer to 0.1 cm, 0.25 cm, 0.5, cm, 075 cm or 1 cm from the proximal end of the circlip. Each possibility is a separate embodiment.

According to some embodiments, the grasping member may include a tooth grasping member.

As used herein, the term “tooth grasping member” may refer to a structure configured to grasp, cover, hold, be glued to or otherwise engage a tooth or a plurality of teeth.

According to some embodiments, the tooth grasping member may include a ring structure configured to circumferentially engage a tooth. According to some embodiments, the tooth grasping member may include an adhesive cover configured to cover a tooth.

According to some embodiments, the tooth grasping member may include a compartment configured to store therein medicaments. According to some embodiments, the compartment may be configured to release the medicaments based on the measured concentration of CO₂. According to some embodiments, the compartment may be configured to release the medicaments based on instructions received from the control logic. According to some embodiments, the compartment may be configured to automatically release the medicaments based on the measured concentration of CO₂.

According to some embodiments, there is provided a patient interface including a grasping member configured to grasp a patient's nose. According to some embodiments, the grasping member may include a first and a second arm, and an elastic member connecting the first and second arms. According to some embodiments, the elastic member may be configured to surroundingly engage a nasal ridge of the patient.

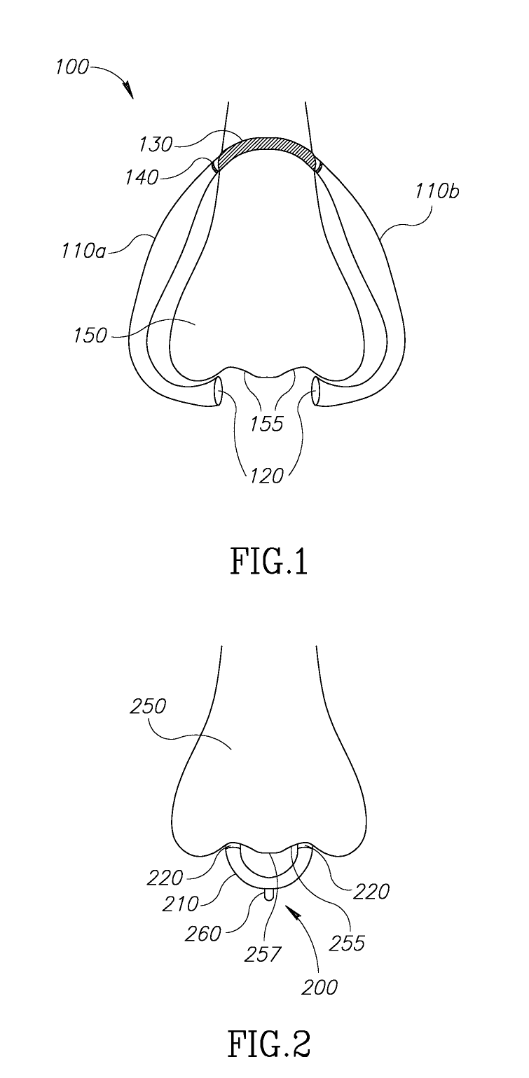

Reference is now made to FIG. 1 which schematically illustrates a tubeless patient interface, according to some embodiments. Patient interface 100 includes grasping arms 110a and 110b connected to one another through elastic member 130, thereby enabling wearing patient interface 100 on a nose 150, similarly to wearing glasses. Grasping arms 110a and 110b each include a miniature CO₂ sensor 120 at proximal ends of grasping members 110a and 110b, as essentially described herein. Grasping members 110a and 110b may terminate right beneath the nostrils 155 of nose 150. Alternatively, grasping members 110a and 110b may enter nostrils 155 (option not shown), for example by 0.25 cm, 0.5 cm, 0.75 cm or 1 cm. Each possibility is a separate embodiment. Miniature CO₂ sensor 120 is configured to measure the concentration of CO₂, directly from the breath flow of the patient, underneath or within nostrils 155. Elastic member 130 is configured to be stretched when worn on nose 150, thereby enabling on/off detection of interface 100. Elastic member 130 includes a SpO2 probe 140 configured to monitor the oxygen saturation of the patient.

Reference is now made to FIG. 2 which schematically illustrates a tubeless patient interface, according to some embodiments. Patient interface 200 includes circlip 210 configured to grasp columella 257 of nose 250 from each side thereof (within each of nostril 255). Circlip 210 includes a miniature CO₂ sensor 220 at each of proximal ends of circlip members 210, as essentially described herein. Miniature CO₂ sensor 220 may thus measure the concentration of CO₂, directly from the breath flow of the patient, within nostrils 255. Patient interface 200 may optionally include a stabilizing element 260 configured to stabilize circlip 200 within nostrils 255 by engaging with the upper lip of the patient (not shown).

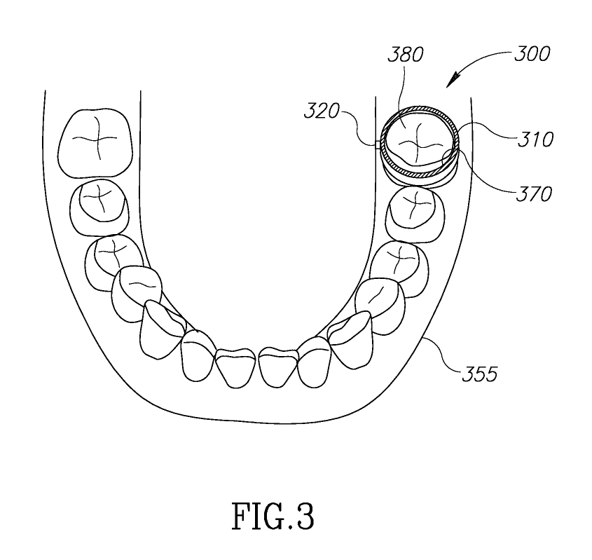

Reference is now made to FIG. 3 which schematically illustrates a tubeless patient interface, according to some embodiments. Patient interface 300 includes a grasping member, ring structure 310 configured to engage a tooth such as tooth 380 of the patient. Ring structure 310 includes a miniature CO₂ sensor 320 configured to measure the concentration of CO₂, directly from the breath flow of the patient, within mouth 355 of the patient. Optionally, ring structure 310 may include a compartment 370 configured to contain therewithin medicaments. Compartment 370 may be formed integrally with ring structure 310, for example compartment 370 may be formed as a hollow channel within ring structure 370. However, other configurations are also possible and as such fall within the scope of the disclosure. For example compartment 370 may be a separate compartment molded on or otherwise attached to ring structure 310 (option not shown). Compartment 370 may be configured to release the medicaments directly into the mouth of the patient based on the measurements obtained from miniature CO₂ sensor 320, as essentially described herein.

It is understood, by one of ordinary skill in the art, that the patient interfaces described herein may be used alone or in combination. For example, the same patient may be sampled from the nose by a nasal patient interface, such as patient interfaces 100 or 200 and from the mouth by an oral patient interface, such as patient interface 300.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope. CLAMS

1. A tubeless patient interface comprising:
a grasping member configured to grasp a patient's nose or tooth; and
a miniature CO₂ sensor attached to said grasping member and configured to measure the concentration of CO₂ from a patient's breath flow.

2. The interface of claim 1, configured to facilitate non-diverted breath sampling.

3. The interface of claim 1, further configured to wirelessly provide the measured CO₂ concentration to a remote control logic.

4. The interface of claim 1, wherein said miniature CO₂ sensor is a nano-sized optical sensor.

5. The interface of claim 1, wherein said miniature CO₂ sensor is a chemical sensor.

6. The interface of claim 1, wherein said grasping member comprises a first and a second arm, and an elastic member configured to connect said first and second arms.

7. The interface of claim 6, wherein said interface is configured to be worn on said patient's nose.

8. The interface of claim 6, wherein said patient interface comprises at least two miniature CO₂ sensors; and wherein each of said first and second arms comprises at least one miniature CO₂ sensor on a proximal end thereof.

9. The interface of claim 8, wherein said proximal ends of said first and second arms are configured to reach underneath or within a patient's nostrils.

10. The interface of claim 6, wherein stretching of said elastic member is indicative of said interface being worn by said patient.

11. The interface of claim 10, wherein said interface is further configured to on/off patient detection based on said stretching of said elastic member.

12. The interface of claim 6, wherein said elastic member comprises an SpO₂ probe.

13. The interface of claim 1, wherein said grasping member comprises a circlip configured to grasp a columella of said nose on each side thereof.

14. The interface of claim 13, said patient interface comprises at least two miniature CO₂ sensors positioned on ends of said circlip.

15. The interface of claim 1, wherein said grasping member is a tooth grasping member.

16. The interface of claim 15, wherein said tooth grasping member comprises a ring structure configured to circumferentially engage a tooth.

17. The interface of claim 15, wherein said tooth grasping member comprises an adhesive cover configured to be attached to a tooth.

18. The interface of claim 15, wherein said tooth grasping member further comprises a compartment configured to store therein medicaments.

19. The interface of claim 18, wherein said compartment is configured to release said medicaments based on the measured concentration of CO₂.

20. A patient interface comprising a grasping member configured to grasp a patient's nose; said grasping member comprising a first and a second arm, and an elastic member connecting said first and second arms; wherein said elastic member is configured to surroundingly engage a nasal ridge of the patient.
